(19) United States
(12) Patent Application Publication
(10) Pub. No.: US 2013/0245075 A1
(43) Pub. Date: Sep. 19, 2013

(54) MELANOGENESIS INHIBITION-METHODS AND COMPOSITIONS THEREOF

(71) Applicants: Muhammed Majeed, Edison, NJ (US); Kalyanam Nagabhushanam, East Windsor, NJ (US)

(72) Inventors: Muhammed Majeed, Edison, NJ (US); Kalyanam Nagabhushanam, East Windsor, NJ (US)

(21) Appl. No.: 13/834,188

(22) Filed: Mar. 15, 2013

Related U.S. Application Data
(60) Provisional application No. 61/612,448, filed on Mar. 19, 2012.

(51) Int. Cl. A61K 31/4412

(52) U.S. Cl. CPC A61K 31/4412
USPC 514/350

(57) ABSTRACT

The present invention relates to melanogenesis inhibition using an effective amount of piper longumine obtained from the roots of Piper longum.

This application is a non-provisional filing of provisional application No. 61/612448 filed on Mar. 19, 2012 the contents of which are incorporated herein by reference.

### FIELD OF INVENTION

The present invention in general relates to compositions and methods for melanogenesis inhibition (reducing skin hyper pigmentation). In specific, the present invention relates to melanogenesis inhibition using an effective amount of piperlongumine obtained from the roots of Piper longum.

### BACKGROUND OF THE INVENTION

### Description of Prior Art

Alkaloids piperlongumine and piperlonguminine from the roots of Piper longum L. are reported in prior art A Chatterjee and C P Dutta, “Alkaloids of Piper longum Linn.-I. Structure and synthesis of piperlongumine and piperlonguminine (Tetrahedron 1967) Volume: 23, Issue: 4, Pages: 1769-1781). Different structural characteristics define the two individual alkaloids, namely piperlongumine and piperlonguminine.

The melanogenesis inhibitory activity of piperlonguminine from the fruits of Piper longum has been demonstrated in prior art (Min K R et al in Planta Med. 2004 and Kim K S et al in Pigment Cell Res. 2006).

Anti-cancer therapeutic potential of piperlongumine (piplartine) for carcinoma, sarcoma and melanoma has been documented before (US20090312373). Other aspects of therapeutic potential for piperlongumine like antiplatelet aggregation, anti-nociceptive, anxiolytic, anti-atherosclerotic, anti-diabetic, anti-bacterial, anti-fungal, leishmanicidal, trypanocidal, and schistosomicidal activities have been documented by Bexerra D P et al, “Overview of the therapeutic potential of piplartine (piperlongumine)”, Eur J Pharm Sci. 2012 Dec 11; 48(3):453-463.

The present invention discloses the efficacy of piperlongumine as a melanogenesis inhibitory agent.

Accordingly, it is the principle objective of the present invention to provide a method of melanogenesis inhibition using piperlongumine.

It is also another objective of the present invention to provide a method for reducing skin hyper pigmentation using piperlongumine.

The present invention fulfills the aforesaid objectives and provides further related advantages.

### SUMMARY OF THE INVENTION

Disclosed are methods for (i) melanogenesis inhibition and (ii) reducing skin hyper pigmentation using effective amounts of piperlongumine.

Other features and advantages of the present invention will become apparent from the following more detailed description which illustrates by way of example, the principle of the invention.

### DETAILED DESCRIPTION OF THE MOST PREFERRED EMBODIMENT

In the most preferred embodiment, the present invention relates to a method for melanogenesis inhibition, said method comprising step of bringing into contact melanocytes and an effective amount of piperlongumine.

In another most preferred embodiment, the present invention relates to a method for reducing skin hyper pigmentation, said method comprising step of bringing into contact melanocytes and an effective amount of piperlongumine.

In another most preferred embodiment, the present invention relates to a method for melanogenesis inhibition, said method comprising step of treating the subject in need thereof with an effective amount of piperlongumine. In a further specific embodiment, the subject is a mammal.

In another most preferred embodiment, the present invention relates to a method for reducing skin hyper pigmentation, said method comprising step of treating the subject in need thereof with an effective amount of piperlongumine. In a further specific embodiment, the subject is a mammal.

The melanogenesis inhibitory potential of piperlongumine is experimentally demonstrated in the underlying illustrative example.

### EXAMPLE 1

A. Principle:

The intracellular melanin in B16 F1 mouse melanoma cells treated with varying concentrations of the sample is extracted by 1N NaOH. The brown colored melanin thus extracted is estimated at 405 nm. This brown color intensity of the melanin is quenched in the presence of the inhibitor.

B. General Methodology:

B16F1 mouse melanoma cells are cultured for 24 hours in a 6 well microtiter plate at a seeding density of 5000 cells per well. Melanin production is induced by α-MSH/cAMP after which the cells are treated with varying non-cytotoxic concentrations of the sample over a period of 9 days with sample treatment at regular intervals of 3 days. The melanin is extracted by 1N NaOH and the absorbance read at 405 nm in a Microplate reader. The inhibitory effect of the sample is calculated based on the degree of inhibition of melanin formation.

C. Calculation:

The results are expressed as IC50 values using Graph pad prism software. The percentage of inhibition of melanin is calculated as follows

% Inhibition=C−T/C×100

Where, C-absorbance due to melanin in untreated cells; T-absorbance due to melanin in sample treated cells

D. Result:
IC₅₀ of piperlongumine was 0.625 μg/ml
IC₅₀ of Kojic acid was 100 μg/ml.

The efficacy of piperlongumine as a melanogenesis inhibitory agent as exemplified in the aforesaid working example demonstrates its cosmeceutical potential. It is evident that piperlongumine could be used to inhibit melanogenesis or to reduce pigmentation in mammals affected by an increased production of melanin pigment.

While the invention has been described with reference to a preferred embodiment, it is to be clearly understood by those skilled in the art that the invention is not limited thereto. Rather, the scope of the invention is to be interpreted only in conjunction with the appended claims

We claim:

1. A method for melanogenesis inhibition, said method comprising step of bringing into contact melanocytes and an effective amount of piperlongumine.

2. A method for melanogenesis inhibition, said method comprising step of treating the subject in need thereof with an effective amount of piperlongumine.

3. The method as claimed in claim 2, wherein the subject is a mammal.

4. A method for reducing skin hyper pigmentation, said method comprising step of bringing into contact melanocytes and an effective amount of piperlongumine.

5. A method for reducing skin hyper pigmentation, said method comprising step of treating the subject in need thereof with an effective amount of piperlongumine.

6. The method as claimed in claim 5, wherein the subject is a mammal.
